(19) United States
(12) Patent Application Publication
(10) Pub. No.: US 2024/0042190 A1
(43) Pub. Date: Feb. 8, 2024

(54) VALVE AND ASSEMBLY FOR CONTROLLED DELIVERY OF MEDICAL FLUIDS

(71) Applicant: Peter Salat, Calgary (CA)

(72) Inventor: Peter Salat, Calgary (CA)

(21) Appl. No.: 17/438,494

(22) PCT Filed: Apr. 30, 2021
(86) PCT No.: PCT/IB2021/053617

§ 371 Date: Sep. 13, 2021

(51) Int. Cl. A61M 39/22; A61M 5/31

(52) U.S. Cl. CPC A61M 39/223; A61M 5/3135; A61M 2039/229

(57) ABSTRACT

A valve and system for controlled delivery of medical fluids to a patient from two or more syringes can be manipulated by a one hand of an operator. The valve includes syringe ports for connecting two or more syringes to the valve and a stop cock that is operable by a handle to selectively connect one syringe at a time to a needle or catheter for delivery of medical fluid to the patient. The valve is configured such that an operator can use his or her the thumb for either operation of the syringe plungers for injection of the liquid contained in the syringe or alternatively for the operation of the valve handle to select which syringe will be operational.

### FIELD OF THE INVENTION

The present invention relates to the delivery of medical fluids to patients by syringes and, more particularly, to a valve for the selected delivery of medical fluids to a patient from two or more syringes by a single-handed operation.

### BACKGROUND OF THE INVENTION

Delivery of medication to a target space or tissue in the body is required for management of the symptoms and treatment of various disease processes, for example the delivery of injectable medication used to treat the symptoms of osteoarthritis. Delivery of medication into the target space is essential for optimal function of the medication and for optimal clinical benefit to the patient. Delivery of medication into a target space/tissue in the body can be achieved through the skin with a needle and syringe combination for superficial targets. For superficial target medication injection, the miss rate can be up to 20% using clinical landmarking for needle guidance.

The accuracy of injection can be improved with image guidance for the placement of the needle. Deeper targets in the body typically require imaging guidance with CT, fluoroscopy or less commonly MM. Access to deep tissues in the body can also be achieved through the vascular system with the use of a catheter and wire combination, for example during the delivery of chemotherapeutic medication for the treatment of liver cancer. Almost any target space in the body can be reached accurately, safely, and rapidly with the use of medical imaging guidance of a needle or catheter/wire combination. Superficial spaces are typically well visualized with ultrasound, while deeper spaces can be accessed with fluoroscopy, CT, or MM.

A rate limiting step during any type of medication injection procedure is the need to switch syringes attached to the needle or catheter. Typically, several medications are injected during a procedure. Initially, the operator will inject a local anesthetic under the skin, then advance the needle into the target space or tissue and switch the syringe containing anesthetic for another syringe containing the medication of interest. This is then injected into the target space or tissue.

For certain procedures, the operator may need to inject another medication or a contrast agent to confirm the position of the needle or catheter within the target. This generally requires another syringe exchange and adds to the length of the procedure. For some procedures, serial syringe exchanges are required to complete the procedure as for example during trans-vascular trans-catheter embolization of a liver tumor. The syringe exchanges add to the time required for the procedure, which is known to be inversely related to patient satisfaction with a procedure. In addition, serial syringe exchanges can add to the risk of a procedure.

For image guided procedures, the operator may require the use of one hand for the operation of the imaging device, for example an ultrasound probe. The requirement to switch syringes or use the second hand to operate a valve switching between syringes necessitates the operator to put down the transducer probe, thus losing sight of the needle and target. The time required for switching the syringe or a valve with the other hand not only delays the completion of the procedure but introduces the potential for displacement or misplacement of the needle, which had been carefully positioned into a target. For trans-vascular procedures, this syringe switching introduces additional risk to the procedure.

This is related to the fact that the catheter connection with the syringe must be without air, which can be a source of unwanted embolization. There is always a small chance of air bubbles collecting in the system when a syringe exchange is performed. A careful purging of the system must be performed and a carefully performed “wet to wet” connection of the syringe and catheter, limiting the chance for air bubbles but adding to the technical complexity and introducing the potential for error. Should the needle or catheter become dislodged from the target while the syringe is being exchanged, which can occur when the scale of the target is small and very subtle motion introduced during the handling of the needle or catheter/syringe combination during a syringe exchange could dislodge the needle or catheter. In such a case, the target will need to be acquired again under image guidance before the target is injected with the desired medication, prolonging the procedure, and increasing the risk to the patient.

### SUMMARY OF THE INVENTION

Embodiments of invention are designed to benefit the operator and the patient regarding the risks described above. The benefits arise from the fact that embodiments of the invention provide a valve and assembly system that can be held and operated with one hand to switch between syringes and to deposit the contents of the syringes or use a syringe for aspiration. This enables the operator freedom to use the other hand to hold for example an ultrasound probe enabling image guidance during the procedure, or to manipulate a catheter.

The ability to continuously monitor the position of the needle or catheter during the procedure is thus enabled—the operator may switch between syringes attached to the valve to select the desired syringe for operation with the same hand that he or she holds the entire assembly. The procedure can thus be performed faster and safer.

In embodiments, the invention is designed such that it enables operation of the valve with the thumb of the hand holding the entire assembly, including the valve and the syringes connected to the valve. The ability to control the valve position while holding the entire assembly in one hand is a unique feature not available with any of the prior devices. This feature is essential for the benefits of the current valve and system in embodiments of the invention.

In embodiments, the design feature of the valve that enables one handed control of the assembly (needle-valve-syringe or catheter-valve-syringe) and operation of the valve handle is that the handle extends to the base of the syringes, where the operator holds the assembly by the syringe finger tabs with the index and middle fingers. With this design, the operator can use the thumb for either operation of the syringe plunger for injection of the liquid contained in the syringe cylinder or alternatively for the operation of the valve handle to select which syringe will be operational.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there are illustrated embodiments of the invention.

### BRIEF DESCRIPTION OF THE DRAWINGS

The following drawings illustrate by way of example and are included to provide further understanding of the invention for the purpose of illustrative discussion of the embodiments of the invention. No attempt is made to show structural details of the embodiments in more detail than is necessary for a fundamental understanding of the invention, the description taken with the drawings making apparent to those skilled in the art how the several forms of the invention may be embodied in practice. Identical reference numerals do not necessarily indicate an identical structure. Rather, the same reference numeral may be used to indicate a similar feature of a feature with similar functionality. In the drawings:

FIG. 1 is diagrammatic top view of a valve and system in accordance with embodiments of the invention;

FIG. 2 is a diagrammatic side view of the valve and system of FIG. 1;

FIG. 3 is a diagrammatic cross-sectional view of the valve of FIG. 1, showing the valve in one position;

FIG. 4 is a diagrammatic cross-sectional view of the valve of FIG. 1, showing the valve in a second position;

FIG. 5 is a diagrammatic cross-sectional view of a valve in accordance with another embodiment of the invention;

FIG. 6 is a diagrammatic side view of a valve and system in accordance with embodiments of the invention;

FIG. 7 is a diagrammatic top view of the valve and system of FIG. 6;

FIG. 8 is a diagrammatic end view of the valve of FIG. 6;

FIG. 9 is a diagrammatic cross-sectional view of the valve of FIG. 6, showing the valve in one position; and

FIG. 10 is a diagrammatic cross-sectional view of the valve of FIG. 6, showing the valve in a second position.

### DETAILED DESCRIPTION OF THE INVENTION

The detailed embodiments of the present invention are disclosed herein. It should be understood, however, that the disclosed embodiments are exemplary of the invention, which may be embodied in various forms. Therefore, the details disclosed herein are not to be interpreted as limiting, but merely as a basis for teaching one skilled in the art how to make and/or use the invention.

Reference throughout this specification to “one embodiment” or “an embodiment” means that particular feature, structure, or characteristic described in connection with the embodiment is included in at least one embodiment. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more embodiments.

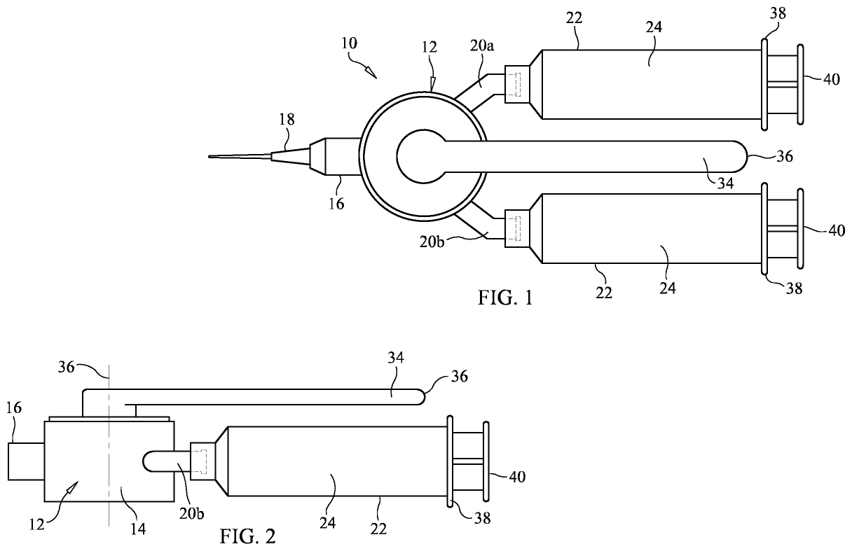

FIGS. 1 through 4 illustrate a valve and syringe assembly 10, according to one example embodiment. The assembly 10 may, for example, be used to provide intra-articular injections by an operator using a single hand to hold and manipulate the assembly. The assembly 10 includes a valve 12 that having a valve body 14, representatively shown having a cylindrical, hub shape. The valve body 14 has an needle orifice or needle port 16 to which a needle 18 is removably connected. While not shown, needle 18 could be replaced by a catheter. The needle port 16 may be formed by or part of a Luer-Lock connector or coupling. For example, the needle port may be part of a male Luer-Lock portion and the needle 18 may include a female Luer-Lock portion that detachably rotatingly couples the with the male Luer-Lock portion.

The valve body 14 further includes, as representatively shown in this embodiment, two inlet orifices or ports 20a and 20b to which syringes 22 are removably connected. The syringe ports 20a and 20b may be formed by or part of a Luer-Lock connector or coupling. For example, the syringe ports 20a and 20b may each be part of a female Luer-Lock portion and the syringes 22 may include a male Luer-Lock portion that detachably rotatingly couples the with the female Luer-Lock portion.

Importantly, each of the syringe ports 20a and 20b are configured such that the barrels 24 of the syringes 22, when the syringes are connected to a respective syringe port, are disposed in a spaced, parallel relation to each other. Further, the syringe ports 20a and 20b are disposed on a diametrically opposite side of the valve body 14 from the needle port 16.

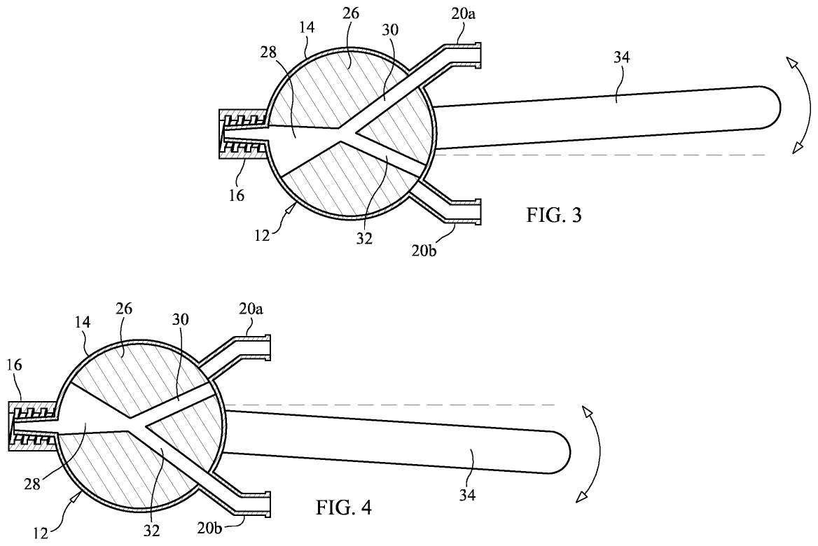

As best seen in FIGS. 3 and 4, the valve 12 further includes a stop cock 26 that is mounted rotatably within the valve body 14. The stop cock 26 includes three flow passages, a distal flow passage 28 and first and second proximal flow passages 30 and 32 with respect to the syringe ports 20a and 20b. The first and second proximal flow passages 30 and 32 are fluidically connected to the distal flow passage 28, in a somewhat V-shape configuration. Further as shown, the outward end of the distal flow passage 28 is frustoconical shaped in cross-section.

The stop cock 26 is rotatable between various states or positions to selectively align only one syringe port 20a or 20b at a time with the needle port 16, while isolating the other syringe port from the needle port. Specifically, with reference to FIG. 3, the stop cock 26 is shown rotated into a position such that flow passage 30 is communicable with syringe port 20a and the needle port 16 to provide fluidic communication between syringe port 20a and needle port 16. In FIG. 4, the stop cock 26 is shown rotated into a position such that flow passage 32 is communicable with syringe port 20b and the needle port 16 to provide fluidic communication between syringe port 20b and needle port 16. The importance of the frustoconical shape of passage 28 is illustrated in FIGS. 3 and 4, wherein passage 28 remains in fluidic communication with the needle port 16 in both shown positions. This configuration helps to maintain a “wet-to-wet” connection when switching between syringes.

The valve 12 further includes a handle 34 that is connected to the stop cock 26 and manipulable to rotate the stop cock. Importantly, the handle 34 is configured to be operated at a distance from axis 36 of the valve 12, such that a thumb or finger can manipulate the valve into the intended position in a patient, opening one port and closing another and depressing or retracting the plunger syringe as needed. Specifically, handle 34 extends outwardly from the valve 12 in the direction in which the syringes 22 extend and terminates at a free end 36. The length of the handle 34 is selected such that the free end 36 thereof is disposed adjacent or in proximity to the finger tabs 38 of the syringes. To this end, valve 12 enables one handed control of the assembly 10 (needle-valve-syringe or catheter-valve-syringe) and operation of the valve handle 34 is that the handle extends to the base of the syringes 22, where the operator holds the assembly by the syringe finger tabs 38 with the index and middle fingers. In the simplest embodiment, the valve handle 34 is positioned such that its long axis travels along the minimum excursion arc along the circumference of the valve body between openings of the individual syringe ports 20a and 20b. With this design, the operator can use the thumb for either operation of the syringe plunger 40 for injection of the liquid contained in the syringe cylinder or alternatively for the operation of the valve handle 34 to select which syringe will be operational.

In embodiments, the arrangement of the syringe ports 20a and 20b for syringe attachment has specific requirements which enable its intended use to be achieved. The limitation on the position of the syringe ports 20a and 20b along the circumference of the valve body 14 must be opposite to the needle port 16 and arranged such that the closest position of the needle ports relative to one another is limited by the cross-sectional diameter of the syringe cylinders 24 attached to the ports (i.e., larger volume syringes have by design larger cross section diameter with limits the position of ports to the distance between the longitudinal axis of the syringe cylinders).

Therefore, the valve can be provided with various configurations of the syringe ports for the use with various diameter syringes. Smaller diameter syringes will be able to be placed closed together while larger caliber syringes will be accommodated by further spacing of the syringe ports along the circumference of the valve body.

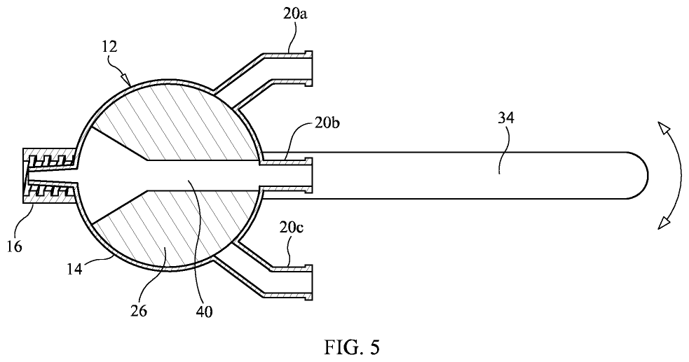

In FIG. 5 there is shown a cross-section of valve 12, according to another example embodiment. Particularly, in this embodiment, valve 12 is configured for attachment with three syringes, rather than two syringes. Here, valve body 14 includes three syringe ports 20a, 20b, and 20c. In aspects, the ports are positioned equidistant such that the space along the circumference of the valve body 14 between the first and the second and the second and the third ports is equivalent to the distance from the cross-sectional center of one syringe and its adjacent parallel syringe.

Further, in this embodiment, stop cock 26 includes a single flow passage 40 that is frustoconical shaped at its distal end and cylindrically shaped at is proximal end to be aligned with syringe ports upon turning the stop cock. The stop cock 26 is rotatable between various states or positions to selectively align only one syringe port 20b, or 20c at a time with the needle port 16, while isolating the other syringe ports from the needle port.

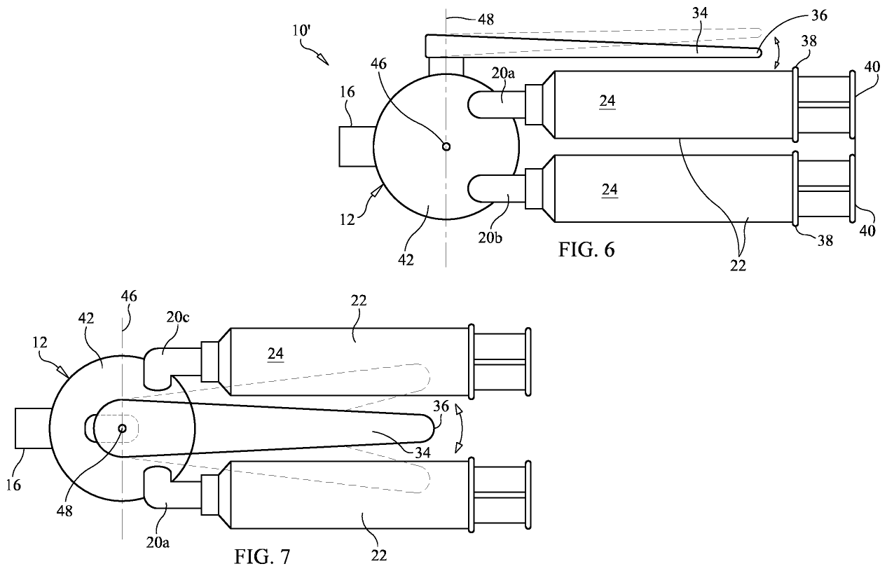

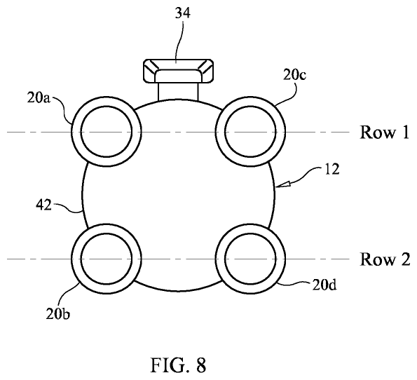

FIGS. 6 through 10 illustrate a valve and syringe assembly 10′, according to another example embodiment, which is constructed and operates like the previously discussed embodiments. In this embodiment, valve body 42 and the stop cock 44 are spherically shaped. As shown, four syringe ports 20a, 20b, 20c, and 20d are provided for attachment to four syringes 22, respectively. The syringe ports are arranged to dispose the syringes in two rows of two syringes. In other aspects, three syringe ports could be provided, and they could be arranged in a triangular pattern.

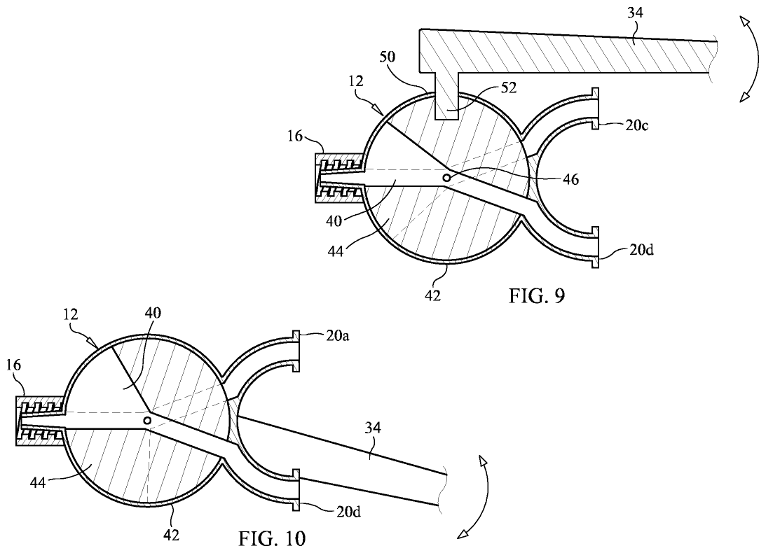

The valve body 42 and stop cock 44 are configured such that the stop cock is movable in two axes of motion as compared to a single axis of motion of the previously described embodiments. Particularly, with reference to FIGS. 6 and 7, handle 34 is pivotably about axis 46 and rotatably about axis 48. In FIG. 9, the stop cock 44 is moved to selectively couple syringe ports in adjacent rows to the needle port by pivoting the handle 34 about axis 46 (extending into the paper). A slot 50 is provided in the valve body 42 to allow the handle stem 52 to move back-and-forth.

As shown in FIG. 9, stop cock 44 is positioned such that flow passage 40 is communicable with syringe port 20d and the needle port 16 to provide fluidic communication between syringe port 20d and needle port 16, while isolating the other three ports. Pivoting handle 34 about axis 46 positions the stop cock 44 such that flow passage 40 is communicable with syringe port 20c and the needle port 16 to provide fluidic communication between syringe port 20d and needle port 16 (shown in dashed line), while isolating the other three ports.

As shown in FIG. 10, stop cock 44 is positioned such that flow passage 40 is communicable with syringe port 20c and the needle port 16 to provide fluidic communication between syringe port 20d and needle port 16, while isolating the other three ports. Pivoting handle 34 about axis 48 positions the stop cock 44 such that flow passage 40 is communicable with syringe port 20a and the needle port 16 to provide fluidic communication between syringe port 20a and needle port 16 (shown in dashed line), while isolating the other three ports.

While the invention has been particularly shown and described with respect to the illustrated embodiments thereof, it will be understood by those skilled in the art that the foregoing and other changes in form and details may be made therein without departing from the spirit and scope of the invention.

What is claimed is:

1. A valve for controlled delivery of medical fluids to a patient from two or more syringes, each syringe having a barrel with a coupling end and a plunger end and finger tabs located at the plunger end, the valve comprising:
a valve body, the valve body having a needle port and at least two syringe ports, the needle port configured to be communicably joined to the patient, each syringe port configured to connect to the coupling end of a syringe to be communicably joined therewith, and wherein each syringe port being arranged such that when syringes are connected to the syringe ports, the barrels of the syringes are disposed parallel to each other;
a stop cock mounted rotatably within the valve body, the stop cock having at least one flow passage and being rotatable between various states to selectively align only one syringe port at time with the needle port for fluidic communication therebetween, while isolating the remaining syringe ports from fluidic communication with the needle port;
a handle attached to the stop cock and extending outwardly from the valve body and terminating at a free end, the handle having a length such that the free end is disposed adjacent the finger tabs of the syringes when the syringes are connected to respective syringe ports, thereby allowing an operator to hold the valve body and the syringes in one hand and operate the handle by thumb of the same hand to selectively fluidically couple each syringe, one at a time, to the needle port.

2. The valve of claim 1, wherein the at least two syringe ports comprise two syringe ports.

3. The valve of claim 1, wherein the at least two syringe ports comprise three syringe ports.

4. The valve of claim 1, wherein the valve body is spherically shaped, the stop cock is spherically shaped, and the at least two syringe ports comprise four syringe ports.

5. The valve of claim 1, wherein the valve body is spherically shaped, and the stop cock is spherically shaped.

6. A system for controlled delivery of medical fluids to a patient, the system comprising:
a plurality of syringes, each having a barrel with a coupling end and a plunger end, and finger tabs disposed at the plunger end;
a valve body, the valve body having a needle port and at least two syringe ports, the needle port configured to be communicably joined to the patient, each syringe port connected to the coupling end of a syringe to be communicably joined therewith, and wherein each syringe port being arranged such that the syringes extending from the valve body with the barrels of each syringe in parallel relation to each other;
a stop cock mounted rotatably within the valve body, the stop cock having at least one flow passage and being rotatable between various states to selectively align only one syringe port at time with the needle port for fluidic communication therebetween, while isolating the remaining syringe ports from fluidic communication with the needle port; and a handle attached to the stop cock and extending outwardly from the valve body and terminating at a free end, the handle having a length such that the free end is disposed adjacent the finger tabs of syringes when the syringes are connected to respective syringe ports, thereby allowing an operator to hold the valve body and the syringes in one hand and operate the handle by thumb of the same hand to selectively fluidically couple each syringe, one at a time, to the needle port.

7. The valve of claim 6, wherein the at least two syringe ports comprise two syringe ports.

8. The valve of claim 6, wherein the at least two syringe ports comprise three syringe ports.

9. The valve of claim 6, wherein the valve body is spherically shaped, the stop cock is spherically shaped, and the at least two syringe ports comprise four syringe ports.

10. The valve of claim 6, wherein the valve body is spherically shaped, and the stop cock is spherically shaped.
